(19) United States
(12) Patent Application Publication
(10) Pub. No.: US 2024/0009156 A1
(43) Pub. Date: Jan. 11, 2024

(54) METHODS AND FORMULATIONS FOR PRENATAL TREATMENT OF ALLAN-HERNDON-DUDLEY SYNDROME

(71) Applicants: PriZm, LLC, Chandler, AZ (US); The University of Miami, Coral Gables, FL (US); The University of Chicago, Chicago, IL (US)

(72) Inventors: Samuel REFETOFF, Chicago, IL (US); Roy WEISS, Miami Beach, FL (US); Khemraj HIRANI, Chandler, AZ (US)

(21) Appl. No.: 18/350,089

(22) Filed: Jul. 11, 2023

Related U.S. Application Data
(60) Provisional application No. 63/388,239, filed on Jul. 11, 2022.

(51) Int. Cl. A61K 31/192; A61P 5/14

(52) U.S. Cl. CPC A61K 31/192; A61P 5/14

(57) ABSTRACT

The present disclosure is directed to methods of treating Allan-Herndon-Dudley syndrome comprising administering 3,5-diiodothyropropionic acid (DITPA) to a pregnant mother of a prenatal subject in need thereof, and to pharmaceutical DIPTA formulations for administration to the pregnant mother of a prenatal subject in need thereof.

### CROSS-REFERENCE TO RELATED APPLICATION

This application claims the benefit of U.S. Provisional Patent Application Ser. No. 63/388,239, filed on Jul. 11, 2022, the content of which is incorporated herein by reference in its entirety.

### TECHNICAL FIELD

The present disclosure is directed to methods of prenatal treatment of Allan-Herndon-Dudley syndrome comprising administering 3,5-diiodothyropropionic acid (DITPA) to pregnant mothers of subjects in need thereof at specific gestation times.

### BACKGROUND ART

Allan-Herndon-Dudley Syndrome (“AHDS”) is an X-linked recessive developmental disorder causing intellectual disability and movement issues in males. Specifically, patients with AHDS have a mutant SLC16A2 gene resulting in a malformed monocarboxylate transporter 8 (“MCT8”) protein. Symptoms of AHDS are caused by a lack of cellular uptake of the thyroid hormone triiodothyronine (“T3”), which is normally transported across the cell membrane by MCT8. This MCT8 deficiency leads to a lack of T3 in tissues that need T3 to function properly contributing to an accumulation of T3 in the blood serum. The other thyroid hormone thyroxine (“T4”) usually remains at normal serum levels in AHDS patients but may also be slightly reduced from a normal level. Thyroid stimulating hormone (“TSH”) is normal to slightly elevated in AHDS patients.

Currently, no treatment for AHDS has been approved by the United States Food and Drug Administration. Clinical trials have been completed for the drug, triiodothyroacetic acid (“TRIAC”), for use in the treatment of AHDS. However, TRIAC shares a close structural similarity to T3, which makes it difficult to accurately assess T3 serum levels. Further, TRIAC has been shown to significantly reduce T4 serum levels. See, Groeneweg et al. Lancet Diabetes Endocrinol. 2019 Sep;7(9);695-706.

3,5-diiodothyropropionic acid (“DITPA”) is another thyroid hormone analog that has been studied for treatment of AHDS. However, as mentioned above, DITPA has not yet been approved for use in the treatment of AHDS. This lack of approval may be due to a lack of effective dosing regimens, stable and effective compositions, and extensive pharmacological assessments. While WO/2012/171065, published Dec. 12, 2012, attempts to establish DITPA dosing regimens for AHDS patients, this publication offers only theoretical examples.

Thus, there is a need for specific, stable, and effective prenatal compositions and suitable dosing regimens of DITPA that are effective at prenatal treatment of AHDS and symptoms of AHDS.

### DISCLOSURE

The present subject matter is directed to pharmaceutical compositions comprising 3,5-diiodothyropropionic acid (“DITPA”), and methods for administering such pharmaceutical compositions for prenatal treatment of Allan-Herndon-Dudley syndrome. The methods comprise administration of the DITPA pharmaceutical composition to pregnant mothers of subjects in need thereof, wherein administration begins no more than ten weeks after conception of the subject.

### BRIEF DESCRIPTION OF THE DRAWINGS

FIG. 1 is a graph showing the dose response for DITPA administration to liver in vitro, and its effect on D1 enzymatic activity, i.e., converting T4 to T3.

### DESCRIPTION OF EMBODIMENTS

The Applicant has discovered dosing timings of 3,5-diiodothyropropionic acid (“DITPA”) that are surprisingly effective for the treatment of Allan-Herndon-Dudley Syndrome (“AHDS”).

In one embodiment, the present technology is directed to methods of treating AHDS comprising administering DITPA daily to a pregnant mother of a subject in need thereof wherein administration begins no more than ten weeks after conception of the subject. Administration may be, for example, orally. Oral administration may be, for example, by tablet, or by dispersible tablet for oral suspension. The dosage may be, for example, up to 2.5 mg/kg, based on T3 titration levels.

In another embodiment, administration to pregnant mothers of subjects in need of treatment for AHDS preferably begins no more than nine weeks after conception, more preferably no more than eight weeks after conception, even more preferably no more than seven weeks after conception, yet even more preferably no more than six weeks after conception, yet even more preferably no more than five weeks after conception and most preferably no more than four weeks after conception.

In another embodiment, the present technology is directed to methods of treating Allan-Herndon-Dudley syndrome comprising the following steps:

a) administering DITPA daily at a first dosage for two weeks to a subject in need thereof;

b) administering DITPA daily at a second dosage for two weeks to the subject wherein the second dosage is greater than the first dosage;

c) measuring triiodothyronine (“T3”) serum levels in the subject, wherein if T3 serum levels are normal the second dosage is administered daily;

d) optionally, adjusting daily dosage of DITPA administered to the subject based on T3 serum levels of the subject measured in step c) wherein if the T3 serum levels are too high a third dosage is administered daily wherein the third dosage in greater than the second dosage and wherein if the T3 serum levels are too low a fourth dosage is administered daily wherein the fourth dosage is less than the second dosage; and

e) optionally, measuring T3 serum levels of the subject about 28 days following initial administration of the third or fourth dosage wherein if T3 serum levels are normal the third or fourth dosage is administered daily; and

f) optionally, adjusting daily dosage of DITPA administered to the subject based on T3 serum levels of the subject measured in step e) wherein if the T3 serum levels are too low following daily administration of the third dosage then the subject is administered the second dosage and wherein if the T3 serum levels are too low following daily administration of the fourth dosage then the subject is administered the first dosage daily and wherein if the T3 serum levels are too high following daily administration of the fourth dosage then the subject is administered the second dosage daily.

In a preferred embodiment, the first dosage is about 1 milligram per kilogram of body weight of the subject per day (“mg/kg/day”).

In another preferred embodiment, the second dosage is about 2 mg/kg/day.

In another preferred embodiment, the third dosage is about 2.5 mg/kg/day.

In another preferred embodiment, the fourth dosage is about 1.5 mg/kg/day.

As used herein the term “too high” refers to a T3 serum level that is more than about 15% over T3 serum levels considered normal for the age of the subject.

As used herein the term “too low” refers to a T3 serum level that is more than about 15% under T3 serum levels considered normal for the age of the subject.

As used herein “normal” T3 serum levels by age of the subject is based on levels disclosed in Lem et al., Serum thyroid hormone levels in healthy children from birth to adulthood and in short children born small for gestational age, J Clin Endocrinol Metab, 2012 Sep, 97(9), 3170-8, doi: 10.1210/jc.2012-1759, Epub 2012 Jun 26.

In a preferred embodiment, the daily dosage of DITPA is administered to a subject in need thereof once a day, more preferably the daily dosage of DITPA is divided in two parts and each part is administered every 12 hours and most preferably the daily dosage of DITPA is divided into three parts and each part is administered every 8 hours.

In a preferred embodiment, administration of DITPA occurs via the oral route.

In one embodiment, DITPA may be formulated in a composition comprising DITPA, or a salt thereof, and one or more pharmaceutically acceptable excipients.

In a preferred embodiment, DITPA, or a salt thereof, may present in the pharmaceutical compositions of the present subject matter at a concentration from about 0.001% to about 10% w/w or w/v.

In a preferred embodiment, the one or more pharmaceutically acceptable excipients may be present in the pharmaceutical compositions of the present disclosure at a concentration from about 90% to about 99.999% w/w or w/v.

Pharmaceutically acceptable excipients suitable for use in the present subject matter include, but are not limited to, disintegrants, binders, fillers, plasticizers, lubricants, permeation enhancers, surfactants, sweeteners, sweetness enhancers, flavoring agents and pH adjusting agents.

The term “disintegrants” as used herein refers to pharmaceutically acceptable excipients that facilitate the disintegration of the tablet once the tablet contacts water or other liquids. Disintegrants suitable for use in the present technology include, but are not limited to, natural starches, such as maize starch, potato starch etc., directly compressible starches such as starch 1500, modified starches such as carboxymethyl starches, sodium hydroxymethyl starches and sodium starch glycolate and starch derivatives such as amylose, cross-linked polyvinylpyrrolidones such as crospovidones, modified celluloses such as cross-linked sodium carboxymethyl celluloses, sodium hydroxymethyl cellulose, calcium hydroxymethyl cellulose, croscarmellose sodium, low-substituted hydroxypropyl cellulose, alginic acid, sodium alginate, microcrystalline cellulose, methacrylic acid-divinylbenzene copolymer salts and combinations thereof.

Binders suitable for use in the present technology include, but are not limited to, polyethylene glycols, soluble hydroxyalkyl celluloses, polyvinylpyrrolidone, gelatins, natural gums and combinations thereof.

Fillers suitable for use in the present technology include, but are not limited to, dibasic calcium phosphate, calcium phosphate tribasic, calcium hydrogen phosphate anhydrous, calcium sulfate and dicalcium sulfate, lactose, sucrose, amylose, dextrose, mannitol, inositol and combinations thereof.

Plasticizers suitable for use in the present subject matter include, but are not limited to, microcrystalline cellulose, triethyl citrate, poly-hexanediol, acetylated monoglyceride, glyceryl triacetate, castor oil, and combinations thereof.

Lubricants suitable for use in the present technology include, but are not limited to, magnesium stearate, sodium stearyl fumarate, stearic acid, glyceryl behenate, micronized polyoxyethylene glycol, talc, silica colloidal anhydrous and combinations thereof.

Permeation enhancers suitable for use in the present subject matter include, but are not limited to, precipitated silicas, maltodextrins, β-cyclodextrins menthol, limonene, carvone, methyl chitosan, polysorbates, sodium lauryl sulfate, glyceryl oleate, caproic acid, enanthic acid, pelargonic acid, capric acid, undecylenic acid, lauric acid, myristic acid, palmitic acid, oleic acid, stearic acid, linolenic acid, arachidonic acid, benzethonium chloride, benzethonium bromide, benzalkonium chloride, cetylpyridium chloride, edetate disodium dihydrate, sodium desoxycholate, sodium deoxyglycolate, sodium glycocholate, sodium caprate, sodium taurocholate, sodium hydroxybenzoyal amino caprylate, dodecyl dimethyl aminopropionate, L-lysine, glycerol oleate, glyceryl monostearate, citric acid, peppermint oil and combinations thereof.

Surfactants suitable for use in the present subject matter include, but are not limited to, sorbitan esters, docusate sodium, sodium lauryl sulphate, cetriride and combinations thereof.

Sweeteners suitable for use in the present technology include, but are not limited to, aspartame, saccharine, potassium acesulfame, sodium saccharinate, neohesperidin dihydrochalcone, sucralose, sucrose, dextrose, mannitol, glycerin, xylitol and combinations thereof.

Sweetness enhancers suitable for use in the present technology include, but are not limited to, ammonium salt forms of crude and refined glycyrrhizic acid.

Flavoring agents suitable for use in the present subject matter include, but are not limited to, peppermint oil, menthol, spearmint oil, citrus oil, cinnamon oil, strawberry flavor, cherry flavor, raspberry flavor, orange oil, tutti frutti flavor and combinations thereof.

pH adjusting agents suitable for use in the present formulation include, but are not limited to, hydrochloric acid, citric acid, fumaric acid, lactic acid, sodium hydroxide, sodium citrate, sodium bicarbonate, sodium carbonate, ammonium carbonate, sodium acetate and combinations thereof.

In another preferred embodiment, the pharmaceutical compositions of the present technology do not contain a preservative.

Pharmaceutical compositions of the present technology may be formulated in any dosage form including but not limited to aerosol including metered, powder and spray, chewable bar, bead, capsule including coated, film coated, gel coated, liquid filled and coated pellets, cellular sheet, chewable gel, concentrate, elixir, emulsion, film including soluble, film for solution and film for suspension, gel including metered gel, globule, granule including granule for solution, granule for suspension, chewing gum, inhalant, injectable including foam, liposomal, emulsion, lipid complex, powder, lyophilized powder and liposomal suspension, liquid, lozenge, ointment, patch, electrically controlled patch, pellet, implantable pellet, pill, powder, powder, metered powder, solution, metered solution, solution concentrate, gel forming solution/solution drops, spray, metered spray, suspension, suspension, syrup, tablet, chewable tablet, coated tablet, coated particles in a tablet, film coated tablet, tablet for solution, tablet for suspension, orally disintegrating tablet, soluble tablet, sugar coated tablet, dispersible tablet, tablet with sensor, tape, troche and wafer and extended release and delayed release forms thereof.

In a preferred embodiment, the pharmaceutical compositions of the present technology are in tablet form. In a more preferred embodiment, the pharmaceutical compositions of the present formulation are in a dispersible tablet form. In an even more preferred embodiment, the pharmaceutical compositions of the present formulation are in a water-dispersible tablet form. In a most preferred embodiment, the pharmaceutical compositions of the present formulation are in a water-dispersible tablet form wherein the tablet is scored such that the tablet is dividable into four equal parts.

In a preferred embodiment, when the pharmaceutical compositions of the present technology are in a water-dispersible tablet form the tablet dispersion time is about 70 seconds or less, more preferably about 60 seconds or less and even more preferably about 40 seconds or less, even more preferably about 30 seconds or less, even more preferably about 20 seconds or less, even more preferably about 10 seconds or less and even more preferably about 5 seconds or less.

As used herein the term “pharmaceutically acceptable” refers to ingredients that are not biologically or otherwise undesirable in an oral application.

As used herein, all numerical values relating to amounts, weights, and the like, are defined as “about” each particular value, that is, plus or minus 10%. For example, the phrase “10% w/w” is to be understood as “9% to 11% w/w.” Therefore, amounts within 10% of the claimed value are encompassed by the scope of the claims.

As used herein “% w/w” refers to the weight percent by weight of the total formulation.

As used herein “% w/v” refers to the weight percent by volume of the total formulation.

As used herein the term “effective amount” refers to the amount necessary to treat a subject in need thereof.

As used herein the term “treatment” or “treating” refers to alleviating or ameliorating AHDS or symptoms of AHDS.

As used herein, the term “stable” includes, but is not limited to, physical and chemical stability.

Pharmaceutically acceptable salts of that can be used in accordance with the current subject matter include but are not limited to hydrochloride, dihydrate hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, mesylate, maleate, gentisinate, fumarate, tannate, sulphate, tosylate, esylate, gluconate, glucoronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

Throughout the application, the singular forms “a,” “an,” and “the” include plural reference unless the context clearly dictates otherwise.

The disclosed embodiments are simply exemplary embodiments of the inventive concepts disclosed herein and should not be considered as limiting, unless the claims expressly state otherwise.

The following examples are intended to illustrate the present technology and to teach one of ordinary skill in the art how to use the formulations of this new technology. They are not intended to be limiting in any way.

### EXAMPLES

### Example 1—Dosing Regimen for a Pre-Natal Subject (Prophetic)

### Method

DITPA was administered to a pregnant mother of a male pre-natal subject that had previously tested positive for the SLC16A2 allele correlated with Allan-Herndon-Dudley syndrome at a daily dosage of 1 mg/kg/day divided over three administration spaced 8 hours apart starting at 4 weeks after conception and ending at birth of the subject.

### Results

The dosing regimen would successfully reduce symptoms of AHDS in the newborn subject as compared to affected newborns whose mothers were not treated with DITPA.

### Example 2—Dosing Regimen for a Pediatric Subject (Prophetic)

### Method

3,5-diiodothyropropionic acid (“DITPA”) was administered to a pediatric patient suffering from Allan-Herndon-Dudley Syndrome at a daily dosage of 1 mg/kg/day divided over 25 three administration spaced 8 hours apart for 2 weeks. Following the first 2 weeks, the daily dosage was increased to 2 mg/kg/day for 2 additional weeks. Following the 2 additional weeks, T3 serum levels were assessed. The patient was found to have T3 serum levels more than 15% below normal. The patient was then administered DITPA at a daily dosage of 1.5 mg/kg/day for 28 days at which time T3 serum levels were reassessed. Upon reassessment T3 serum levels were 30 normal.

### Results

The dosing regimen would successfully allow identification of proper dosing for the pediatric patient to maintain normal T3 serum levels.

### Example 3—Dosing Regimen for a Pediatric Subject (Prophetic)

### Method

DITPA was administered to a pediatric patient suffering from Allan-Herndon-Dudley Syndrome at a daily dosage of 1 mg/kg/day divided over three administration spaced 8 hours apart for 2 weeks. Following the first 2 weeks, the daily dosage was increased to 2 mg/kg/day for 2 additional weeks. Following the 2 additional weeks, T3 serum levels were assessed. The patient was found to have T3 serum levels more than 15% above normal. The patient was then administered DITPA at a daily dosage of 2.5 mg/kg/day for 28 days at which time T3 serum levels were reassessed. Upon reassessment T3 serum levels were normal.

### Results

The dosing regimen would allow successful identification of proper dosing for the pediatric patient to maintain normal T3 serum levels.

Example 4—Dosing Regimen for a Pediatric Subject (Prophetic)

### Method

DITPA was administered to a pediatric patient suffering from Allan-Herndon-Dudley Syndrome at a daily dosage of 1 mg/kg/day divided over three administration spaced 8 hours apart for 2 weeks. Following the first 2 weeks, the daily dosage was increased to 2 mg/kg/day for 2 additional weeks. Following the 2 additional weeks, T3 serum levels were assessed. The patient was found to have T3 serum levels more than 15% below normal. The patient was then administered DITPA at a daily dosage of 1.5 mg/kg/day for 28 days at which time T3 serum levels were reassessed. Upon reassessment T3 serum levels were again found to be more than 15% below normal. The patient was then administered DITPA at a daily dosage of 1.0 mg/kg/day for 28 days at which time T3 serum levels were reassessed. Upon reassessment T3 serum levels were found to be normal.

### Results

The dosing regimen would allow successful identification of proper dosing for the pediatric patient to maintain normal T3 serum levels.

### Example 5—In vitro evidence of direct effect of SRW-101 (DITPA) in decreasing the T3 generated from T4: SRW101 (DITPA) inhibiting D1 enzymatic activity in liver in vitro

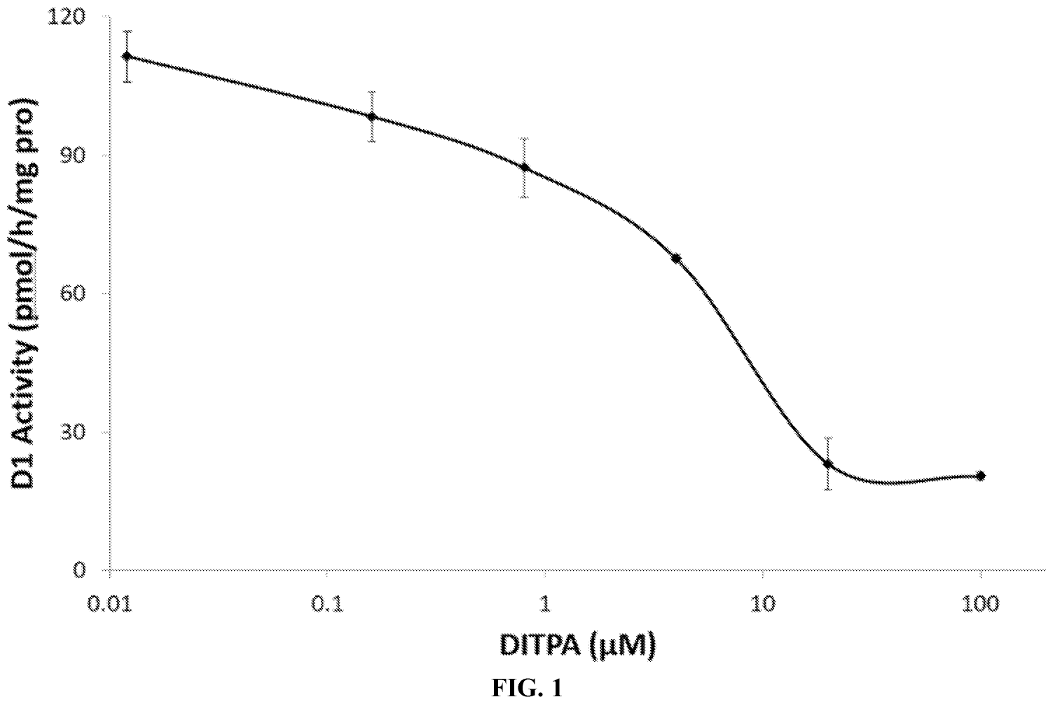

DITPA reduces the activity of deiodinase-1 in vivo and in vitro in liver (see FIG. 1). FIG. 1 reflects the dose response of DITPA added to liver in vitro vs. the measurement of D1 enzymatic activity (i.e., conversion of T4 to T3). This reduction in activity is the main mechanism for reduction in T3 concentration, and increase in T4 concentration, by reducing its consumption and conversion from T4 to T3. It was shown to occur in humans with MCT8 deficiency.

These are the consequences of the normalization of serum T3 levels, a critical endocrine biomarker and parameter for therapeutic efficacy, as they measure the anticipated metabolic changes resulting from the normalization of the thyroid tests.

More specifically, the reduction of T3, which acts on peripheral tissue to accelerate the metabolism, is expected to improve nutrition and increase the ability to gain weight.

Important measurements such as weight gain (corrected for age) and metabolic parameters (cholesterol, creatine kinase, SHBG) are secondary endpoints.

Annotated observations by the parents such as sleep, food record, and motor activity are of immense value.

This provided in vitro evidence of the direct effect of DITPA in decreasing the T3 generated from T4, rather than reducing it through a decrease of T4 by TSH suppression, as is the case with TRIAC. T4 is important to the brain even in the presence of reduced uptake due to MCT8 deficiency.

Effect of DITPA on removal of gastrostomy tube (G-tube)

One child who started DITPA while having a G-tube, gained weight and the g-tube was removed.

Additional information

We have a Planned Clinical Trial for confirmatory evidence to support single-study NDA approval. We designed the proposed Phase 3 study to be robust with endpoints intended to demonstrate the clinical benefit of DITPA versus surrogate endpoints.

Below are the specific endpoints of the planned Phase 3 study and associated rationales:

| FDA | |
|---|---|
| Concurred | |
| Endpoints | Expected Outcome with Justification |
| Neurologic | Studies assessing the neurological and behavioral deficiencies in MCT8- |
| Developmental | mutant (mct82/2) showed that DITPA (SRW101) and other TH analogs |
| (Age | restored the myelin and axon outgrowth deficiencies in mct82/2 larvae |
| Appropriate) | (Zada et al 2014). These studies also showed that the SRW101 and other |
| CHOP- | TH analogs partially rescued the hypomyelination in the CNS of MCT8 |
| INTEND total | mutant (mct8−/−) zebrafish (Zada et al 2016). |
| score | These studies in particular show that administration of SRW101 (and |
| Head Control | other TH analogs) early in infant development can specifically reduce |
| Scale total | neurologic damage in patients with AHDS. It is noted that the TH |
| score | analog Triac has also been studied in pediatric subjects with positive |
| Gross Motor | effects. |
| Function | Zada D, Tovin A, Lerer-Goldshtein T, Vatine G D, and Appelbaum L |
| Measure | (2014). Altered Behavioral Performance and Live Imaging of Circuit- |
| (GMFM)-88 | Specific Neural Deficiencies in a Zebrafish Model for Psychomotor |
| | Retardation. PLoS Genet. 10(9): e1004615. |
| | Zada D, Tovin A, Lerer-Goldshtein T, Vatine G D, and Appelbaum L |
| | (2016). Pharmacological treatment and BBB-targeted genetic therapy |
| | for MCT8-dependent hypomyelination in zebrafish. Disease Models & |
| | Mechanisms. 9, 1339-1348. |
| Endocrine and | Studies assessing the metabolic effects of MCT8 deficiency using |
| Metabolism (I) | MCT8-deficient (knockout) mice (Mct8KO). These studies showed that |
| | SRW101 normalized all measurements and other parameters of TH |
| | action, and that SRW101 is relatively MCT8 independent for entry into |
| | the brain and corrects the TH deficit in Mct8KO mice without causing |
| | thyrotoxic effect in the liver (Di Cosmo et al 2009); and analysis of TH |
| | target genes revealed amelioration of the thyrotoxic state in the liver |
| | (ameliorating hypermetabolism). |
| | Ferrara A M, Liao X, Ye H, Weiss R E, Dumitrescu A M, and Refetoff S |
| | (2015). The thyroid hormone analog DITPA ameliorates metabolic |
| | parameters of male mice with Mct8 deficiency. Endocrinology. |
| | 156: 3889-3894. |
| | Di Cosmo C, Liao X H, Dumitrescu A M, Weiss R E, and Refetoff S |
| | (2009). A thyroid hormone analog with reduced dependence on the |
| | monocarboxylate transporter 8 for tissue transport. Endo. 150(9): 4450- |
| | 4458. |
| Endocrine and | These are the consequences of the normalization of serum T3 levels |
| Metabolism | (first primary endpoint) as they measure the anticipated metabolic |
| (II) | changes resulting from the normalization of the thyroid tests. More |
| | specifically, the reduction of T3, which acts on peripheral tissue to |
| | accelerate the metabolism, is expected to improve nutrition, and increase |
| | the ability to gain weight. |
| | Important measurements such as weight gain (corrected for age) and |
| | metabolic parameters (cholesterol, creatine kinase, SHBG) are |
| | secondary endpoints. |
| | Annotated observations by the parents such as sleep, food record, motor |
| | activity are of immense value. |
| | See FIG. 1: Dose response of DITPA added to liver in vitro and |
| | measurement of the effect on D1 enzymatic activity (conversion of T4 |
| | to T3) in vitro evidence of direct effect of DITPA in decreasing the T3 |
| | generated from T4, rather than reducing it through decrease in T4 by |
| | TSH suppression, as is the case with TRIAC. T4 is important to the |
| | brain even in the presence of reduced uptake due to MCT8 deficiency. |
| Measure of | MCT8-deficient mice have increased energy expenditure and reduced |
| decreasing | fat mass that is abrogated by normalization of serum T3 levels (Di |
| thyrotoxicosis | Cosmo et al 2013). Clinically, children with MCT8 deficiency lose |
| Improvement | weight, even when adequately nourished. Changes in serum markers of |
| in in body | thyroid hormone (TH) action compatible with thyrotoxicosis suggested |
| weight, basal | that this might be due to T3 excess in peripheral tissues. We used |
| metabolic | MCT8-deficient mice as they replicate the human thyroid phenotype and |
| index (BMI) | are thus suitable for metabolic studies that were unavailable in humans. |
| Avoidance of | As compared to wild-type mice, MCT8KO mice were leaner due to |
| feeding tube | reduced fat mass. They tended to use more carbohydrates and fewer |
| Change in | lipids during the dark phase. MCT8KO mice had increased total energy |
| head | expenditure (TEE) and food and water intake, with normal total activity, |
| circumference | indicating hypermetabolism. To determine whether this is due to the |
| and | high serum T3, we studied mice deficient in both MCT8 and deiodinase |
| Decrease in | 1 (Mct8D1KO) with serum T3 similar to wild type mice and wilt type |
| frequency of | mice given L-T3 to raise their serum T3 to the level of Mct8KO mice. |
| dyskinetic | Contrary to MCT8KO, MCT8D1KO mice had similar fat mass, TEE, |
| episodes. | and food intake as their DIKO littermates, whereas T3-treated wild type |
| | mice showed increased food intake and TEE, similar to MCT8KO mice. |
| | In skeletal muscle, MCT8KO mice had increased T3 content and TH |
| | action and increased glucose metabolism, which improved in |
| | MCT8D1KO mice. These studies indicate that the high serum T3 in |
| | MCT8 deficiency increases the TEE and fails to maintain weight despite |
| | adequate calorie intake. This is mediated by tissues that are not |
| | predominantly MCT8 dependent for TH transport, including skeletal |
| | muscle. Normalizing serum T3 level by deleting deiodinase 1 corrects |
| | body composition and the metabolic alterations caused by the MCT8 |
| | deficiency (Di Cosmo et al 2013). |
| | Di Cosmo C, Liao X H, Ye H, Ferrara A M, Weiss R E, Refetoff |
| | S, and Dumitrescu A M (2013). Mct8-deficient mice have |
| | increased energy expenditure and reduced fat mass that is |
| | abrogated by normalization of serum T3 levels. Endocrinology. |
| | 154, 4885-4895. |

Our primary endpoint was chosen to ensure high probability of NDA success based on following factors:

Our estimated PTRS for reaching primary endpoint based on T3 level difference at the end of randomized withdrawal period has >99% power to detect a change of at least 100 ng/dL in serum T3 levels from baseline (start of randomized withdrawal) to week 8 (week 34 of trial) vs. placebo. We know from prior studies (such as LT3 treatment in primary hyperthyroidism) that LT3 levels increase within hours after treatment and therefore in the 8-week period T3 levels in MCT8 deficient patients off SRW101 treatment should have ample time increase sharply and return to baseline high within days.

The key secondary endpoint is to assess the complete total T3, free T4, and TSH response rate at the end of the dose-titration and maintenance treatment with SRW-101 in the initial single-arm, open label part of the study (Week 24) in the mITT population. The key secondary null hypothesis is that the proportion of patients who are total T3, free T4, and TSH complete responders at the Week 24 Visit is less than or equal to 0.2 The alternative hypothesis is that the proportion of patients who are total T3, free T4, and TSH complete responders at the Week 24 Visit is greater than 0.2. The exact test for one proportion will be used. Efficacy of SRW-101 will be declared when the proportions of responders at the Week 24 Visit is statistically significantly greater than 0.2 at a one-sided alpha level of 0.025. A sample size of 40 patients age 0-17 years will have nearly 100% power to detect a difference of 100 ng/dL using a one-sided exact test for one proportion with a target significance level of 0.025. For the secondary outcomes, it is assumed that the population proportion under the null hypothesis is 0.2 and the alternative hypothesis is 0.80.

The number and proportions (expressed as percentages) of total T3, free T4, and TSH responders at each scheduled time point during the OLDT period and OLDM period, including the Week 24 Visit will be calculated. These proportions, along with their exact (Clopper-Pearson) 95% CIs, will be summarized by scheduled time point. Enrolled patients who had missing thyroid function test assessment at Week 24 will be counted as non-responders for the key secondary endpoint. Other secondary endpoint analyses will be specified in the statistical analysis plan (SAP) and approximate powers will be calculated then.

It is to be understood that the subject matter herein is not limited to the specific embodiments described above but encompasses any and all embodiments within the scope of the generic language of the following claims enabled by the embodiments described herein, or otherwise described above in terms sufficient to enable one of ordinary skill in the art to make and use the claimed subject matter.

We claim:

1. A method of treating Allan-Herndon-Dudley syndrome, the method comprising determining that a prenatal subject is in need of treatment for Allan-Herndon-Dudley syndrome;
and administering 3,5-diiodothyropropionic acid (DITPA) or a salt thereof to a pregnant mother of the prenatal subject,
wherein administration begins no more than ten weeks after conception of the subject.

2. The method of claim 1, wherein administration to the pregnant mother begins no more than nine weeks after conception.

3. The method of claim 1, wherein administration to the pregnant mother begins no more than eight weeks after conception.

4. The method of claim 1, wherein administration to the pregnant mother begins no more than seven weeks after conception.

5. The method of claim 1, wherein administration to the pregnant mother begins no more than six weeks after conception.

6. The method of claim 1, wherein administration to the pregnant mother begins no more than five weeks after conception.

7. The method of claim 1, wherein administration to the pregnant mother begins no more than four weeks after conception.

8. The method of claim 1, wherein the administering comprises administration to the pregnant mother of a daily dose.

9. The method of claim 8, where the daily dose is administered in one dose, once daily.

10. The method of claim 8, where the daily dose is divided in two parts administered twice daily or every 12 hours.

11. The method of claim 8, where the daily dose is divided in three parts administered three times daily or every 8 hours.

12. The method of claim 1, where the administration comprises oral administration.

13. A pharmaceutical composition for treating Allan-Herndon-Dudley syndrome in a prenatal subject in need of such treatment, the composition comprising 3,5-diiodothyro-propionic acid (DITPA) or a salt thereof.

14. The pharmaceutical composition of claim 13, wherein the composition is formulated for oral administration.

15. The pharmaceutical composition of claim 13, wherein the composition is formulated for daily administration.

16. The pharmaceutical composition of claim 13, wherein the composition further comprises one or more pharmaceutically acceptable excipients.

17. The pharmaceutical composition of claim 13, wherein the DITPA or salt thereof is present at a concentration from about 0.001% to about 10% w/w or w/v.

18. The pharmaceutical composition of claim 16, wherein the one or more pharmaceutically acceptable excipients is present at a concentration from about 90% to about 99.999% w/w or w/v.

19. The pharmaceutical composition of claim 18, wherein the one or more pharmaceutically acceptable excipients includes at least one of the following: disintegrants, binders, fillers, plasticizers, lubricants, permeation enhancers, surfactants, sweeteners, sweetener enhancers, flavoring agents, and pH adjusting agents.
